(19) United States
(12) Patent Application Publication
(10) Pub. No.: US 2013/0071871 A1
(43) Pub. Date: Mar. 21, 2013

(54) ENZYMATIC DETERMINATION OF LITHIUM IONS USING PHOSPHOGLUCOMUTASE

(71) Applicants: Richard A. Kaufman, Manville, NJ (US); Peter I. Màthé, Franklin Park, NJ (US)

(72) Inventors: Richard A. Kaufman, Manville, NJ (US); Peter I. Màthé, Franklin Park, NJ (US)

(73) Assignee: SPECIALTY ASSAYS, INC., Hillsburough, NJ (US)

(21) Appl. No.: 13/238,784

(22) Filed: Sep. 21, 2011

(51) Int. Cl. C12Q 1/32; C12Q 1/533

(52) U.S. Cl. 435/26; 435/4

(57) ABSTRACT

Kits and methods for measuring lithium ions using phosphoglucomutase are disclosed.

### TECHNICAL FIELD

The invention is directed to the use of phosphoglucomutase to measure lithium ions in samples.

### BACKGROUND OF THE INVENTION

Lithium ions are used clinically in the treatment of manic depressive psychosis. Lithium exerts its therapeutic effect by producing a sedative effect on components of the central nervous system. The therapeutic effect of lithium, however, is only effective over a rather narrow serum concentration range, from about 0.8 to 1.2 mmol/L (Fundamentals of Clinical Chemistry, Norbert W. Tietz, 3rd. ed., 1987, p. 863). If the serum level is too low (<0.8 mmol/L), no or little therapeutic effect is observed, and if the serum level is too high (>1.2 mmol/L), severe toxic side effects can occur. Therefore, it is essential that the serum level of lithium ions be monitored for lithium to exert its therapeutic effect.

Methods used to measure lithium ions in body fluids include flame emission photometry, atomic absorption spectrophotometry, phosphatase enzyme inhibition (Diazyme, San Diego, Calif. 92186), porphyrin binding (U.S. Pat. No. 7,241,623 B2), crown ethers (U.S. Pat. Nos. 5,344,782 and 5,344,782) and lithium-ion specific electrodes. Given the potential fire hazards of the use of open flames in clinical laboratories more laboratories are switching to the non-flame methods of measuring lithium in clinical samples.

Although the above procedures are generally effective, some are potentially hazardous (flame photometric methods), expensive (crown ethers), use toxic reagents (modified porphyrin) or require rather expensive instrumentation (ion-specific electrodes). Thus, there exists a need for a cheaper, less toxic alternative method for the measurement of lithium ions in clinical laboratory samples.

### SUMMARY OF THE INVENTION

In accordance with one aspect of the invention, the present invention includes a diagnostic reagent kit for measuring lithium ions in a sample. Broadly speaking, the kit includes a) a cofactor such as NAD(P) or an NAD(P) analog, and b) phosphoglucomutase. Preferably, the kit further includes glucose-1-phosphate and glucose-6-phosphate dehydrogenase. Optionally, the kits can include glucose-1-6-diphosphate, a metal ion source such as magnesium sulfate, a metal binding agent such as malonic acid and a buffer in sufficient amount and strength to keep the pH at about 6 to 9. In some preferred aspects of the invention, the kits are designed to include at least two containers so that in a first container there is included the cofactor, glucose-1-phosphate, metal ion source, metal binding agent and buffer, while the second container includes the phosphoglucomutase, glucose-6-phosphate dehydrogenase, additional metal ion source and buffer.

In accordance with another embodiment of the invention, there are provided methods of quantifying the presence of lithium ions in a sample. The methods include:
a) contacting a sample containing lithium ions with a first reagent which comprises an NAD(P) or NAD(P) analog cofactor mentioned above, glucose-1-phosphate, and optionally one or more of glucose-1,6-diphosphate, a buffer, a metal binding agent and a metal ion source;
b) adding a second reagent which comprises glucose-6-phosphate dehydrogenase and phosphoglucomutase, and optionally a buffer and a metal ion source to the mixture resulting from step a) and
c) measuring the change in absorbance or fluorescence resulting from step b).

Preferred NAD(P) analogs are thio-NAD and thio-NADP. A preferred metal ion source is magnesium sulfate. A. preferred metal binding agent is malonic acid. A preferred buffer is TRIS, having a pH of from about 6 to about 9.

As a result of the present invention there are provided kits and methods for measuring the presence of lithium in a sample which have cost and safety advantages over the prior art.

### DETAILED DESCRIPTION OF THE INVENTION

The enzyme phosphoglucomutase is known to convert glucose-1-phosphate to glucose 6-phosphate by means of a pathway which is presented in a simplified form below. Glucose-6-phosphate can be utilized as a substrate by the enzyme glucose-6-phosphate dehydrogenase in conjunction with the oxidized form of nicotinamide adenine dinucleotide (phosphate) (NAD (P)) or analogs thereof to yield glucolactone and the reduced form of the nucleotide (NAD(P)H). This latter reaction provides an easy method of determining the activity of phosphoglucomutase. See the following sequence of reactions:

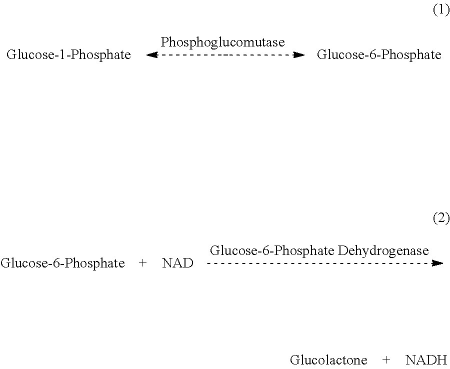

The reaction in which NAD is reduced to NADH is followed by measuring an increase in absorbance at approximately 340 nm. In this reagent system, glucose-6-phosphate must first be formed from glucose-1-phosphate by means of phosphoglucomutase.

The presence and quantification of lithium ions in clinical samples can be determined by measuring the inhibition of phosphoglucomutase using the coupled reaction sequence in reactions (1) and (2) above. In this coupled reaction sequence, the activity of phosphoglucomutase is followed by measuring the rate of formation of glucose-6-phosphate using glucose-6-phosphate dehydrogenase and NAD to produce gluconate and the NADH which is measured at 340 nm. It will be appreciated that NAD is used as an illustrative example of NAD, NADP, and synthetic analogs thereof, rather than in a limiting form.

As an alternative enzyme cofactor, NADP or analogs of NAD or NADP can also be used for better sensitivity. Some examples of suitable NAD and NADP analogs include S-NAD, Ac-NAD, Ald-NAD, S-NADP, Ac-NADP, Ald-NADP, Deamino-NAD and Deamino-NADP. Alternatively, the cofactor analogs can be selected from among, for example, 3-acetylpyridine adenine dinucleotide, 3-acetylpyridine adenine dinucleotide phosphate, thionicotinamide adenine dinucleotide, thionicotinamide adenine dinucleotide phosphate, 3-pyridinealdehyde adenine dinucleotide, 3-pyridinealdehyde adenine dinucleotide phosphate, nicotinamide hypoxanthine dinucleotide and nicotinamide hypoxanthine dinucleotide phosphate. Preferred analogs include thionicotinamide adenine dinucleotide (phosphate), i.e. thio-NAD and thio-NADP. In some alternative embodiments, the cofactor analogs of NAD and NADP are those disclosed in commonly assigned U.S. Pat. No. 6,380,380 to Kaufman, the contents of which are incorporated herein by reference. The analogs can be prepared using known techniques or purchased from Specialty Assays Inc, of Hillsborough, N.J.

Lithium reagents using phosphoglucomutase as the lithium ion concentration indicator can be manufactured as stable liquid reagents with long shelf life. In addition, the kit components of suitable lithium ion kits are inexpensive and most components are readily available. Typical components included in kits prepared in accordance with the present invention include two-reagent systems which contain the NAD(P) or NAD(P) analog, phosphoglucomutase, and suitable buffers among other components, such as, optionally, glucose-6-phosphate dehydrogenase, and glucose-1-phosphate. The concentration of the NAD(P) or NAD(P) analogs in the reagent kits can range from 0.1 mmol/L to about 3.0 mmol/L.

The phosphoglucomutase included in the kits of the present invention is preferably obtained from yeast. One particularly preferred phosphoglucomutase enzyme is obtained from the yeast species Saccharomyces cerevisiae. The phosphoglucomutase from the yeast species Saccharomyces cerevisiae is one preferred enzyme to use in the assay. The enzyme is stable at elevated temperatures, and is particularly sensitive to lithium. Other sources of phosphoglucomutase may be known to those of ordinary skill in the art. Useful enzyme activities in the final reagent range from 5 units/L to 1000 units/L, with a preferred activity of about 30 to 150 units/L. The enzyme can be extracted using known techniques or purchased from Specialty Assays Inc. of Hillsborough, N.J. In alternative embodiments, the enzyme is obtained from other yeasts or other sources in accordance with known techniques. The invention is not limited to the sources of the enzyme described herein for illustrative purposes.

The diagnostic reagent kits of the present invention can also include a divalent metal ion cofactor such as magnesium or manganese. Preferably, the metal ion cofactor is magnesium, which is preferably included in the kits as magnesium sulfate or similar salts for the metal ion source. The divalent metal ion cofactor is needed to activate phosphoglucomutase and glucose-6-phosphate dehydrogenase. Magnesium is the known natural activator, and so is preferred. Additional magnesium, coming from the sample to be tested for lithium, will elevate the activity of phosphoglucomutase unless it is in some way buffered.

The kits may also include a metal chelator to bind and buffer additional metals such as magnesium coming from the sample to be tested. Malonic acid is one preferred metal binding agent. Alternatives include but are not limited to non-lithium acids of citrate, (i.e. citric acid), succinate (succinic acid), phosphonate (phosphonic), oxalate (oxalic), phosphate (phosphoric), etc. In assays in accordance with the present invention, the metal chelator, e.g. malonic acid is used for the purpose of buffering this excess magnesium coming from the sample. Other metal chelators with similar effects will be known to those of ordinary skill in the art.

In accordance with the present invention, any buffer can be used to maintain the pH of the reagent in a range of about 6 to 9; however, one particularly preferred buffer is TRIS (tris(hydroxymethyl)amino-methane). Although preferred buffering systems include TRIS, other suitable buffers can be used with a pKₐ of about 6.5 to 8.5. A non-limiting list of such buffers include but are not limited to: ADA (N-(2-Acetamido)iminodiacetic acid), carbonate, ACES (N-(2-Acetamido)-2-aminooehtanesulfonic acid), PIPES (piperazine-N,N-bis(2-ethanesulfonic acid)), MOPSO (3-(N-Morpholino)propanesulfonic acid), imidazole, BIS-TRIS (Bis(2-hydroxyethyl)imino-tris(hydroxymethyl)methane), BES (N,N-Bis(hydroxyethyl)-2-aminoethanesulfonic acid), MOPS (3-(N-Morpholino)-2-hydroxypropanesulfonic acid), HEPES (N-(2-Hydroxyethyl)piperazine-N′-(2-ethanesulfonic acid)), TES (N—[Tris(hydroxymethyl)methyl]-2-aminoethanesulfonic acid), methylimidazole, DIPSO (3-[N,N-Bis(hydroxyethyl)amino]-2-hydroxypropanesulfonic acid), TAPSO(N—[Tris(hydroxymethyl)methyl]-3-amino-2-hydroxypropanesulfonic acid), triethanolamine, pyrophosphate, HEPPSO(N-(2-hydroxyethyl)piperazine-M-2-hydroxypropanesulfonic acid), POPSO (piperazine-N,N′-bis(2-hydroxypropanesulfonic acid)), tricine, glycylglycine, EPPS(N-(2-Hydroxyethyl)piperazine-N′-3-propanesulfonic acid), and BICINE ((2-hydroxyethyl)glycine).

The buffer concentration can range from about 0.01 to 0.5 mol/L, and, in the case of TRIS, the preferred concentration is about 0.083 mol/L. The reagent solutions containing the NAD(P) or NAD(P) analog have a pH in the range of about 6 to about 9. A pH in the range of about 7 to about 8.5 is more preferred, with a most preferred pH of about 7.8.

Further optional components included in the kits of the present invention include a substrate for phosphoglucomutase, glucose-1-phosphate. The most readily available sodium salt form is preferred, though other forms known to those of ordinary skill are be acceptable. The kits can also include glucose-6-phosphate dehydrogenase. While most commercially available forms of glucose-6-phosphate dehydrogenase will be acceptable for use in this assay, it has been determined that the enzyme from Leuconostoc mesenteroides works particularly well and is quite stable at elevated temperatures, e.g., at 41° C.

The kits of the present invention can also include from about a 50 to about a 1,000 nanomol/L concentration of glucose-1,6-diphosphate which functions to assure that the phosphoglucomutase is sufficiently activated.

The diagnostic reagent kits of the present invention preferably include a two-reagent system in which there are provided two separate containers. This is known as a two-reagent configuration. For the sake of simplicity, the first container holds the first reagent, and the second container holds the second reagent. Reagent 1 will have a pH in the range about 6 to about 9, and preferably 7.8. The second reagent, Reagent 2, will also have a pH in the range about 6 to about 9, preferably 7.5. When Reagents 1 and 2 are combined the pH will be in the range about 6 to about 9, and preferably about 7.8.

In one preferred aspect on the invention, the first reagent, Reagent 1, preferably includes:

a. TRIS buffer, pH 7.8;

b. malonic acid;

c. magnesium sulfate;

d. thio-NAD; and

e. glucose-1-phosphate.

The second reagent, Reagent 2, in this preferred aspect of the invention preferably includes:

a. TRIS buffer, pH 7.5;

b. magnesium sulfate;

c. glucose-6-phosphate dehydrogenase; and

d. phosphoglucomutase.

In an alternative embodiment, there is provided a diagnostic reagent kit for measuring lithium ions or the presence of lithium in a sample. The kit includes

a) a first container having therein
i) a cofactor selected from the group consisting of NAD (nicotinamide adenine dinucleotide), NADP (nicotinamide adenine dinucleotide phosphate), NAD analogs and NADP analogs;
ii) glucose-1-phosphate; and
iii) glucose-1,6-diphosphate, and
iv) optionally one or more of a buffer, a metal binding agent and a metal ion source; and

b) a second container having therein
i) glucose-6-phosphate dehydrogenase;
ii) phosphoglucomutase; and
iii) optionally one or more of a buffer and a metal ion source.

A preferred method of the present invention to measure lithium ion concentration is the inhibition of phosphoglucomutase in the following series of coupled enzymatic reactions:

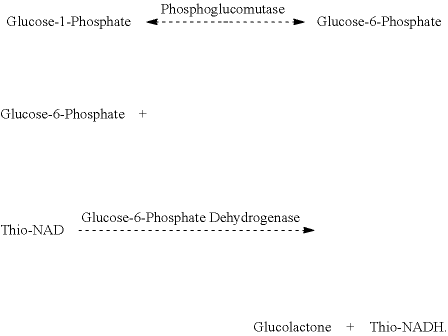

In this procedure, yeast phosphoglucomutase will convert glucose-1-phosphate in two steps to glucose-6-phosphate by means of a phosphoenzyme, with glucose-1,6-diphosphate as an intermediate (not shown). As glucose-6-phosphate becomes available, glucose-6-phosphate dehydrogenase will convert this substrate to glucolactonephosphate while simulateneously reducing thio-NAD to thio-NADH. The rate of increase in absorbance due to the formation of thio-NADH can be measured at wavelengths between 350 and 440 nm, with maximum sensitivity at about 405 nm. The concentrion of lithium ions, which are known to inactivate phosphoglucomutase, is determined by measuring the rate of formation of thio-NADH. Calibration is performed on samples without lithium and with an elevated level of lithium, resulting in a calibration curve from which sample values can then be interpolated.

The kits of the present invention can be prepared in either wet or dry form, including lyophilized form, depending upon the needs of the user. If desired, the kits can be prepared to include a suitable antimicrobial compound such as sodium azide, which can be present in amounts up to 0.5% by weight.

The kits of the present invention can also be prepared to include enzyme stabilizers such as bovine serum albumin and sorbitol; the former can be used in amounts up to 1%, the latter up to 40%, by weight.

To perform the assay, a sample containing no lithium (“0” calibrator) is used to determine the baseline (non-inhibited) activity of phosphoglucomutase and then a sample containing an elevated level of lithium, e.g. about 3.0 mmol/L, is used as an “elevated lithium standard” to determine the phosphoglucomutase activity with a known lithium ion concentration. Then patient samples with intermediary lithium levels can then be interpolated from this calibration in accordance with techniques well known to those of ordinary skill.

In accordance with another embodiment of the invention, there are provided methods for determining lithium ion concentration in a sample. The methods include:
a) contacting a sample containing lithium ions with a first reagent which comprises a cofactor selected from the group consisting of NAD (nicotinamide adenine dinucleotide), NADP (nicotinamide adenine dinucleotide phosphate), NAD analogs and NADP analogs, glucose-1-phosphate; and optionally one or more of glucose-1,6-diphosphate, a buffer, a metal binding agent and a metal ion source;
b) adding a second reagent which comprises glucose-6-phosphate dehydrogenase and phosphoglucomutase, and optionally a buffer and a metal ion source to the mixture resulting from step a) and
c) measuring the change in absorbance or fluorescence resulting from said step b).

The methods of the invention will measure/read the change in absorbance over a wavelength of about 340 to about 440 nm.

In accordance with these method aspects of the invention, the NAD(P) analogs can be selected from among 3-acetylpyridine adenine dinucleotide, 3-acetylpyridine adenine dinucleotide phosphate, thionicotinamide adenine dinucleotide, thionicotinamide adenine dinucleotide phosphate, 3-pyridinealdehyde adenine dinucleotide, and 3-pyridinealdehyde adenine dinucleotide phosphate, nicotinamide hypoxanthine dinucleotide and nicotinamide hypoxanthine dinucleotide phosphate. Other analogs not specifically named but known to those of ordinary skill can also be used if desired in the methods and kits described herein. As was the case with the kits, one preferred NAD analog is thionicotinamide adenine dinucleotide while one NADP analog is thionicotinamide adenine dinucleotide phosphate.

Similar to the kits described herein, the methods are preferably carried out with the enzyme phosphoglucomutase is obtained from a yeast species, preferably Saccharomyces cerevisiae. The divalent metal ion included in the method is magnesium, the metal binding agent is preferably is malonic acid. The buffer selected for use in the method preferably has a pH of from about 6 to about 9 and is more preferably about 7.8. One particularly preferred buffer is TRIS.

Within one preferred aspect of the invention, the first reagent, i.e. Reagent 1, includes: TRIS buffer, pH 7.8; malonic acid; magnesium sulfate; thio-NAD; glucose-1-phosphate; and glucose-1,6-diphosphate. The second reagent, i.e. Reagent 2, includes: TRIS buffer, pH 7.5; magnesium sulfate; glucose-6-phosphate dehydrogenase; and phosphoglucomutase.

A related method of the invention includes a first and a second container. The first container having therein
i) a cofactor selected from the group consisting of NAD (nicotinamide adenine dinucleotide), NADP (nicotinamide adenine dinucleotide phosphate), NAD analogs and NADP analogs;
ii) glucose-1-phosphate; and
iii) glucose-1,6-diphosphate, and
iv) optionally one or more of a buffer, a metal binding agent and a metal ion source; and
   the second container having therein
i) glucose-6-phosphate dehydrogenase;
ii) phosphoglucomutase; and
iii) optionally one or more of a buffer and a metal ion source.

### EXAMPLES

The following non-limiting example illustrates certain aspects of the invention. All parts and percentages are by weight unless otherwise noted and all temperatures are in degrees Celsius. The principal chemicals were obtained from commercial suppliers such as Sigma-Aldrich Co., the NAD(P) analogs were produced by Specialty Assays Inc., the phosphoglucomutase was produced by Specialty Assays Inc., and the glucose-6-phosphate dehydrogenase was purchased from Toyobo Co., Ltd.

### Example 1

The reagent is configured as a two component liquid-stable reagent. The following is an example of a two-component reagent for measuring lithium ions in samples. There are many variations of this formulation which would be acceptable, as those skilled in the art of developing reagents will recognize.

| | Acceptable | | Preferred | |
|---|---|---|---|---|
| Reagent 1 | Range | | Range | |
| Buffer with pH of 7.8 | pH 6-9 | | pH 7-8.5 | |
| (TRIS preferred at | | | | |
| 0.1 mol/L) | | | | |
| 0.1 mol/L TRIS buffer | 0.01-0.5 | mol/L | 0.05-0.25 | mol/L |
| 0.125 mol/L malonic acid | 0.01-0.5 | mol/L | 0.05-0.25 | mol/L |
| 1.7 mmol/L thio-NAD | 0.1-3.0 | mmol/L | 0.5-2.5 | mmol/L |
| 6 mmol/L magnesium sulfate | 0.1-50 | mmol/L | 1.0-20 | mmol/L |
| 5.33 mmol/L glucose-1- | 0.1-50 | mmol/L | 1.0-20 | mmol/L |
| phosphate | | | | |
| 0.3% bovine serum albumin | 0-10% | | 0.01-1% | |
| 0.05% sodium azide | 0-5% | | 0.01-1% | |
| 0.13 μmol/L glucose- | 0-100 | μmol/L | 0.05-0.5 | μmol/L. |
| 1,6-disphosphate | | | | |

| | Acceptable | | Preferred | |
|---|---|---|---|---|
| Reagent 2 | Range | | Range | |
| Buffer with pH of 7.5 | pH 6-9 | | pH 7-8.5 | |
| (TRIS preferred at | | | | |
| 0.05 mol/L) | | | | |
| 0.05 mol/L TRIS buffer | 0.01-0.5 | mol/L | 0.01-0.25 | mol/L |
| 20 mmol/L magnesium | 0.1-200 | mmol/L | 1.0-100 | mmol/L |
| sulfate | | | | |
| 30% sorbitol | 0-50% | | 5-40% | |
| 0.1% bovine serum | 0-10% | | 0.01-1% | |
| albumin | | | | |
| 0.05% sodium azide | 0-5% | | 0.01-1% | |
| 10,000 units/L glucose-6- | 1000-100,000 | units/L | 2000-20,000 | units/L |
| phosphate dehydrogenase | | | | |
| 500 units/L | 25-5,000 | units/L | 200-1,000 | units/L. |
| phosphoglucomutase | | | | |

### Example 2

The lithium assay is run by adding one volume of sample to fifteen volumes of Reagent 1. After an incubation period, e.g., five minutes, three volumes of Reagent 2 are added plus one volume of diluent (distilled or deionized water). After a short lag phase absorbance readings are taken over a given time period, e.g., five minutes, and the rate of absorbance change per minute at an aforementioned wavelength is calculated.

When the sample, Reagent 1, Reagent 2, and diluent are all combined, the working reagent can have the following final concentrations and activity levels:

82.5 mmol/L TRIS buffer, pH 7.8
93.8 mmol/L malonic acid
1.28 mmol/L thio-NAD
7.5 mmol/L magnesium sulfate
4 mmol/L glucose-1-phosphate
0.24% bovine serum albumin
0.045% sodium azide
4.5% sorbitol
1500 units/L glucose-6-phosphate dehydrogenase
75 units/L phosphoglucomutase
0.1 μmol/L glucose-1,6-disphosphate.

The assay is calibrated using two standards. A protein-based “zero” standard, containing no lithium with physiological levels of sodium, potassium, and magnesium. The protein-based lithium ion standard should be well above the therapeutic range, preferably around a highly toxic level of 3 mmol/L lithium. Both standards should be prepared using human serum or bovine serum albumin.

### Example 3

Below is shown some representative precision data, including the % coefficients of variation and standard deviations, demonstrating the recovery of lithium from serum based Controls.

| | Control 1 | Control 2 | Control 3 |
|---|---|---|---|
| Precision Data: | | | |
| 1 | 0.47 | 1.07 | 1.94 |
| 2 | 0.44 | 1.05 | 1.97 |
| 3 | 0.46 | 1.07 | 1.98 |
| 4 | 0.47 | 1.08 | 1.97 |
| 5 | 0.46 | 1.07 | 1.99 |
| 6 | 0.50 | 1.07 | 1.96 |
| 7 | 0.49 | 1.09 | 1.96 |
| 8 | 0.50 | 1.07 | 1.94 |
| 9 | 0.50 | 1.08 | 1.93 |
| 10 | 0.53 | 1.09 | 1.97 |
| sum | 4.83 | 10.74 | 19.60 |
| avg | 0.48 | 1.07 | 1.96 |
| stdev | 0.0250 | 0.0118 | 0.0201 |
| % cv | 5.18 | 1.10 | 1.02 |
| Assigned control values: | | | |
| atomic abs. | 0.36-0.46 | 0.72-1.34 | 1.72-2.18 |
| mean | 0.41 | 1.03 | 1.95 |
| flame photo. | 0.38-0.48 | 0.82-1.22 | 1.67-1.13 |
| mean | 0.43 | 1.02 | 1.90 |

What is claimed is:

1. A diagnostic reagent kit for enzymatic measurement of lithium ion concentration, comprising a cofactor selected from the group consisting of NAD (nicotinamide adenine dinucleotide), NADP (nicotinamide adenine dinucleotide phosphate), NAD analogs and NADP analogs and phosphoglucomutase.

2. The diagnostic reagent kit of claim 1, further comprising:
a. glucose-1-phosphate;
b. glucose-6-phosphate dehydrogenase; and optionally
c. glucose-1-6-diphosphate.

3. The diagnostic reagent kit of claim 1, wherein the NAD(P) analog is selected from the group consisting of: 3-acetylpyridine adenine dinucleotide, 3-acetylpyridine adenine dinucleotide phosphate, thionicotinamide adenine dinucleotide, thionicotinamide adenine dinucleotide phosphate, 3-pyridinealdehyde adenine dinucleotide, and 3-pyridinealdehyde adenine dinucleotide phosphate, nicotinamide hypoxanthine dinucleotide and nicotinamide hypoxanthine dinucleotide phosphate.

4. The diagnostic kit of claim 3, wherein the NAD analog is thionicotinamide adenine dinucleotide.

5. The diagnostic reagent kit of claim 1, wherein the NADP analog is thionicotinamide adenine dinucleotide phosphate.

6. The diagnostic reagent kit of claim 5, wherein the phosphoglucomutase is from a yeast species.

7. The diagnostic reagent kit of claim 6, wherein the yeast species is Saccharomyces cerevisiae.

8. The diagnostic reagent kit of claim 1, further comprising a divalent metal ion.

9. The diagnostic reagent kit of claim 1, further comprising magnesium sulfate.

10. The diagnostic reagent kit of claim 1, further comprising a metal binding agent.

11. The diagnostic reagent kit of claim 10, wherein the metal binding agent is malonic acid.

12. The diagnostic reagent kit of claim 1, further comprising a buffer.

13. The diagnostic reagent kit of claim 12, wherein the buffer has a pH of about 6 to about 9.

14. The diagnostic reagent kit of claim 13, wherein the buffer has a pH of 7.8.

15. The diagnostic reagent kit of claim 14, wherein the buffer is TRIS.

16. The diagnostic reagent kit of claim 1, further comprising a first container and a second container.

17. The diagnostic reagent kit of claim 16, wherein the first container includes Reagent 1, which comprises:
a. TRIS buffer, pH 7.8;
b. malonic acid;
c. magnesium sulfate;
d. thio-NAD; and
e. glucose-1-phosphate; and
f. glucose-1,6-diphosphate.

18. The diagnostic reagent kit of claim 16, wherein the second container includes Reagent 2, which comprises:
a. TRIS buffer, pH 7.5;
b. magnesium sulfate;
c. glucose-6-phosphate dehydrogenase; and
d. phosphoglucomutase.

19. A diagnostic reagent kit for measuring the presence of lithium in a sample, comprising
a) a first container having therein
i) a cofactor selected from the group consisting of NAD (nicotinamide adenine dinucleotide), NADP (nicotinamide adenine dinucleotide phosphate), NAD analogs and NADP analogs;
ii) glucose-1-phosphate; and
iii) glucose-1,6-diphosphate, and
iv) optionally one or more of a buffer, a metal binding agent and a metal ion source; and
b) a second container having therein
i) glucose-6-phosphate dehydrogenase;
ii) phosphoglucomutase; and
iii) optionally one or more of a buffer and a metal ion source.

20. A method for determining lithium ion concentration in a sample; comprising:
a) contacting a sample containing lithium ions with a first reagent which comprises a cofactor selected from the group consisting of NAD (nicotinamide adenine dinucleotide), NADP (nicotinamide adenine dinucleotide phosphate), NAD analogs and NADP analogs, glucose-1-phosphate; and optionally one or more of glucose-1,6-diphosphate, a buffer, a metal binding agent and a metal ion source;
b) adding a second reagent which comprises glucose-6-phosphate dehydrogenase; and phosphoglucomutase and optionally a buffer and a metal ion source to the mixture resulting from step a) and
c) measuring the change in absorbance or fluorescence resulting from said step b).

21. The method of claim 20, where the change in absorbance is read over a wavelength of about 340 to about 440 nm.
